(19) United States
(12) Patent Application Publication
(10) Pub. No.: US 2005/0059735 A1
(43) Pub. Date: Mar. 17, 2005

(54) Cyclic amino acid derivatives useful as pharmaceutical agents

(76) Inventors: Justin Stephen Bryans, Sandwich (GB); David Clive Blakemore, Sandwich (GB); Sophie Caroline Williams, Cambridge (GB)

Correspondence Address: WARNER-LAMBERT COMPANY, 2800 PLYMOUTH RD, ANN ARBOR, MI, 48105, US

(21) Appl. No.: 10/975,675

(22) Filed: Oct. 28, 2004

Related U.S. Application Data
(63) Continuation of application No. 10/296,355, filed on Apr. 14, 2003.

(30) Foreign Application Priority Data
May 26, 2000 (GB) 0012850.4

(51) Int. Cl. A61K 31/325; A61K 31/215

(52) U.S. Cl. 514/484; 514/529; 514/530

(57) ABSTRACT

Pro-drug compounds of the formula (I) or (II) and compositions containing them are provided that when administered to humans or other mammals provide an increased duration of active compound in the plasma compared to compounds of corresponding structure in which labile groups are not present. 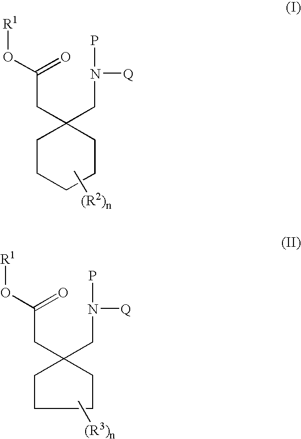
In the above formulae n, P, Q, R¹, R² and R³ are as defined in the specification. The compounds may be used to treat a range of neurological conditions, e.g. epilepsy or pain.

This application is a continuation application of U.S. Ser. No. 10/296,355 filed on Apr. 14, 2004 which is a 371 application of PCT/GB01/02353 filed May 25, 2001, which claims the benefit of Great Britain Patent Application No. 00128350.4 filed May 26, 2000.

### FIELD OF THE INVENTION

This invention relates to novel cyclic amino derivatives useful as pharmaceutical agents, to processes for their production, to pharmaceutical compositions containing them, and to their use for the prevention or treatment of the neurological conditions set out below.

### BACKGROUND TO THE INVENTION

Gabapentin (Neurontin®) is an anti-convulsant agent that is useful in the treatment of epilepsy and that has recently been shown to be a potential treatment for neurogenic pain. It is 1-(aminomethyl)-cyclohexylacetic acid of structural formula:

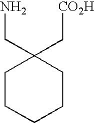

Gabapentin is one of a series of compounds of formula 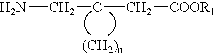
in which R₁ is hydrogen or a lower alkyl radical and n is 4, 5, or 6. These compounds are described U.S. Pat. No. 4,024,175 and its divisional U.S. Pat. No. 4,087,544. Their disclosed uses are: protective effect against cramp induced by thiosemicarbazide; protective action against cardiazole cramp; the cerebral diseases, epilepsy, faintness attacks, hypokinesia, and cranial traumas; and improvement in cerebral functions. The compounds are useful in geriatric patients. The disclosures of the above two patents are hereby incorporated by reference.

WO 97/33858 whose disclosure is incorporated herein by reference describes novel substituted cyclic amino acids, their derivatives, prodrugs and pharmaceutically acceptable salts that are of the formula: 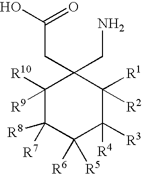
in which R¹ to R¹⁰ are each independently selected from straight or branched chain C¹-C⁶ alkyl, substituted or unsubstituted benzyl or phenyl which substituents are selected from halogen, alkoxy, alkyl, hydroxy, carboxy, carboalkoxy, trifluoromethyl and nitro, any of R¹ to R¹⁰ which is not one of the above being hydrogen. They are useful in the prevention or treatment of epilepsy, faintness attacks, hypokinesia, cranial disorders, neurodegenerative disorders, depression, anxiety, panic, pain and neuropathological disorders.

WO 99/21824, whose disclosure is also incorporated by reference, discloses further cyclic amino acids that are useful in the prevention or treatment of epilepsy, faintness attacks, neurodegenerative disorders, depression, anxiety, panic, pain, neuropathological disorders, digestive disorders such as irritable bowel syndrome, and inflammation, especially arthritis. The compounds disclosed include those of the formula: 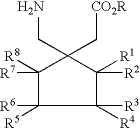
and salts thereof, in which:
R is hydrogen or a lower alkyl;
R¹ to R⁸ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, and R¹ to R⁸ are not simultaneously hydrogen.

The compound methyl N-carbomethoxy-1-aminomethyl-1-cyclohexane-acetate is disclosed as an intermediate in U.S. Pat. No. 4,152,326 and a genus that includes the above compound is also disclosed as an intermediate in WO 99/21824. The compound [1-(t-butoxycarbonylamino-methyl)-cyclohexyl]-acetic acid is disclosed as an intermediate in WO 99/31075. None of the above three references discloses or suggests that the relevant compound has any further utility.

### SUMMARY OF THE INVENTION

A problem with which this invention is concerned is the production of gabapentin analogues that when administered to humans or other mammals provide an increased duration of active compound in the plasma.

That problem is solved, according to the invention, by a pharmaceutical composition comprising a pharmaceutically acceptable carrier and a therapeutically effective amount of a compound of the formula (I) or (II) 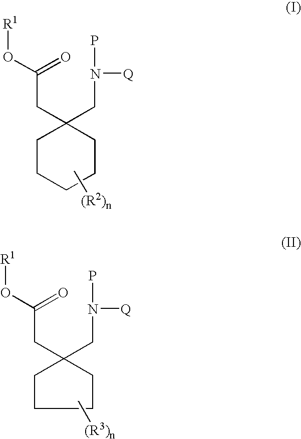
wherein:
n is 0, 1 or 2;
P represents hydrogen or methyl;
Q represents a labile amine- or amide-forming organic group that becomes removed in the human or animal body, and in a compound of formula (I) is other than acyl;
R¹ represents hydrogen or a labile ester-forming group selected from substituted and unsubstituted C₁-C₆ alkyl, benzyl and phenyl groups that become removed in the human or animal body;
R² represents methyl; and
the groups R³ (which when n is 2 may be the same or different) represent C₁-C₆ alkyl, or a pharmaceutically acceptable salt thereof.

Many of the above defined compounds are novel. In a further aspect the invention provides a compound of the formula (I) or (II) 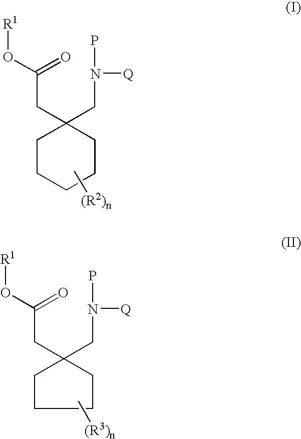
wherein:
n is 0, 1 or 2;
P represents hydrogen or methyl;
Q represents a labile amine- or amide-forming organic group that is selected from 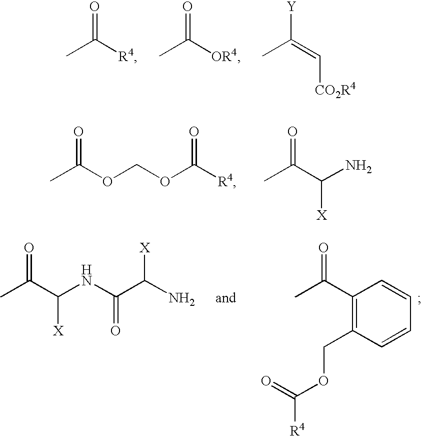
R¹ represents hydrogen or a labile ester-forming group selected from substituted and unsubstituted C₁-C₆ alkyl, benzyl and phenyl groups that become removed in the human or animal body;
R² represents methyl;
the group or groups R³ (which when n is 2 may be the same or different) represent C₁-C₆ alkyl;
R⁴ represents hydrogen, straight or branched chain C₁-C₆ alkyl, phenyl or benzyl in which the benzene ring may be substituted or unsubstituted;
Y represents hydrogen, straight or branched chain C₁-C₆ alkyl, or —CH₂CO₂R⁵ in which R⁵ represents straight or branched chain C₁-C₆ alkyl; and
X represents a phenyl group or any of the side chains of the 20 naturally encoded α-amino acids; or a pharmaceutically acceptable salt thereof
provided that (a) in a compound of formula (I) when Q is —COR⁴ or COOR⁴, R⁴ is not alkyl, and (b) in a compound of formula (II) Q is not —COOMe.

It is believed that pharmaceutical composition as defined above or a pro-drug of the above formula when administered to a human or other mammal enters the bloodstream by passive diffusion along the whole length of the intestine, which gives a much longer duration of effectiveness. The pro-drug may not itself be biologically active, but decomposes to the corresponding active compound in plasma. We have found from a study that a gabapentin amide prodrug administered as a single PO dose to rats gave a similar blood concentration of gabapentin compared to that obtained when gabapentin itself is dosed, but a half-life of over 6 hours compared to 1.2 hours for gabapentin itself.

Certain of the compounds of the invention can exist in unsolvated forms as well as solvated forms, including hydrated forms. In general, the solvated forms, including hydrated forms, are biologically equivalent to unsolvated forms and are within the scope of the invention. Certain of the compounds of the invention possess one or more chiral centers and each center may exist in the R or S configuration. The invention includes all enantiomeric and epimeric forms as well as the appropriate mixtures thereof. It also includes salts of any of the above compounds with physiologically acceptable cations or anions.

The invention also provides a method for making a compound of the formula (I) or (II) above, which comprises:
coupling a compound of the formula: 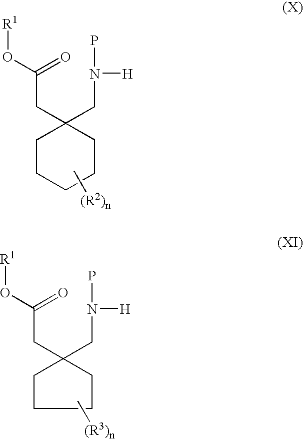
in which P and R¹ have the meanings given above and in which said compound is in the form of a free base or an ammonium salt with a compound of the formula 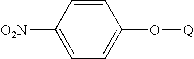
or QCl where Q has the meaning defined above; and
where the compound (X) or (XI) is a carboxylic acid optionally employing the further step of esterifying the carboxyl group with a substituted or unsubstituted C₁-C₆ alkanol, benzyl alcohol or phenol and optionally converting the compound to a physiologically acceptable salt.

The invention also provides a pharmaceutical composition comprising a therapeutically effective amount of a compound according of formula (I) or (II) as aforesaid and a pharmaceutically acceptable carrier.

In a further aspect the invention provides the use of a compound of formula (I) or (II) in the manufacture of a medicament for the prevention or treatment of any of the following:

| | |
|---|---|
| epilepsy; | a faintness attack; |
| hypokinesia; | a cranial disorder; |
| a neurodegenerative disorder; | depression; |
| anxiety; | panic; |
| pain; | a neuropathological disorder; a |
| | digestive disorder. |

In a further aspect, the invention provides a method for preventing or treating any of the above disorders which comprises administering a therapeutically effective amount of a compound of formula (I) or (II) to a mammal in need of said prevention or treatment.

### DESCRIPTION OF PREFERRED FEATURES

One preferred class of compounds of the invention comprises gabapentin pro-drugs of the formula (IIIa) 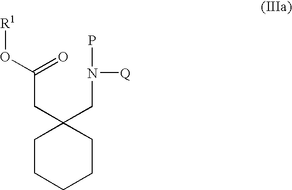
in which R¹, P and Q are as defined above.

Further preferred compounds of the invention comprise pro-drugs of gabapentin analogues disclosed in WO 97/33858, and in particular pro-drugs of compounds disclosed in that specification as having particular activity, for example the compounds of formula (IIIb) and (IIIc): 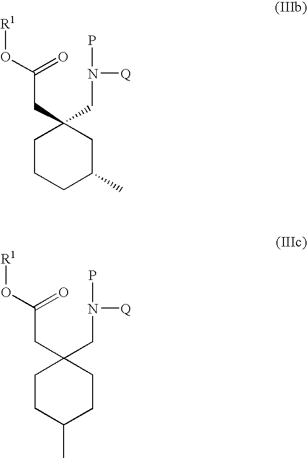
in which R¹, P and Q are as defined above.

In the 5-membered ring compounds of formula (II) the substituents R³ are preferably in the 3- or the 3,4-positions. Yet further compounds of the invention comprise pro-drugs of the compounds disclosed in WO 99/21824 as having particular activity, for example compounds of the formula (IV)-(IX) 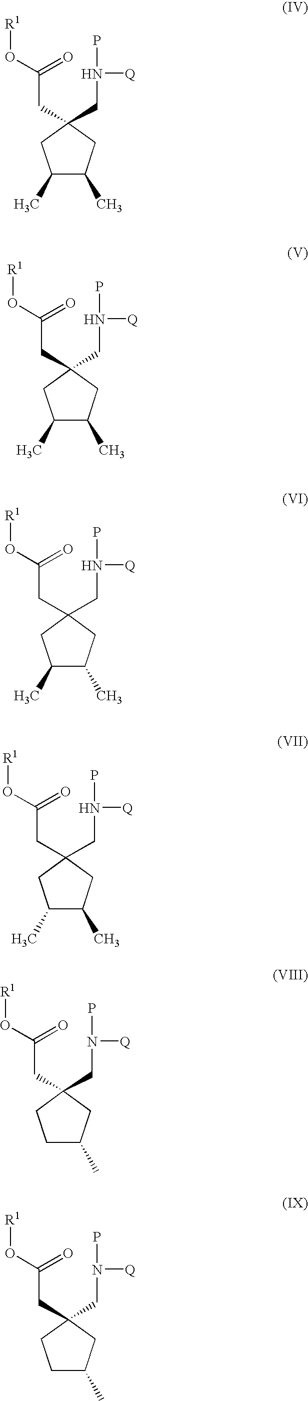
in which R¹, P and Q are as defined above.

As previously stated, the group R¹ may be hydrogen or it may be a group other than hydrogen, in which case it is preferably more labile than Q, specially preferred values being methyl and t-butyl.

Q may be a group that is removable by hydrolysis under physiological conditions, e.g. 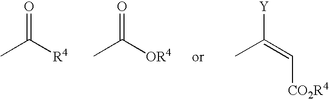
wherein:
R⁴represents hydrogen, straight or branched chain C₁-C₆ alkyl, phenyl or benzyl in which the benzene ring may be substituted or unsubstituted; and
Y represents hydrogen, straight or branched chain C₁-C₆ alkyl, or —CH₂CO₂R⁵ in which R⁵ represents straight or branched chain C₁-C₆ alkyl.

Q may also be a group that is removable by enzymes under physiological conditions, e.g. 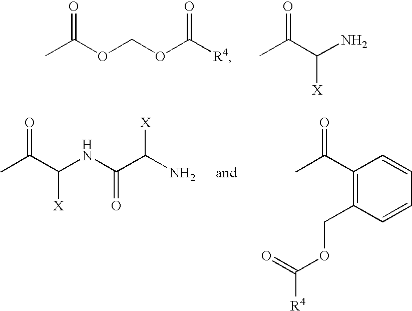
wherein R⁴ represents hydrogen, straight or branched chain C₁-C₆ alkyl, phenyl or benzyl in which the benzene ring may be substituted or unsubstituted (preferably t-butyl, benzyl or phenyl) and X represents a phenyl group or any of the side chains of the 20 naturally encoded α-amino-acids.

Preferred values for R¹ are hydrogen, ethyl, iso-propyl, phenyl or benzyl.

Compounds according to the invention that may be made include the following:
(i) [1-(acetoxymethoxycarbonylamino-methyl)-cyclohexyl]-acetic acid;
(ii) [1-(acetoxymethoxycarbonylamino-methyl)-cyclohexyl]-acetic acid ethyl ester;
(iii) 2,2-dimethyl-propionic acid 1-carboxymethylcyclohexylmethylcarbamoyloxymethyl ester;
(iv) 2,2-dimethyl-propionic acid 1-ethoxycarbonylmethylcyclohexylmethylcarbamoyloxymethyl ester;
(v) benzoic acid 1-carboxymethyl-cyclohexylmethylcarbamoyloxymethyl ester;
(vi) benzoic acid 1-ethoxycarbonylmethylcyclohexylmethylcarbamoyloxy-methyl ester;
(vii) [1-(benzoylamino-methyl)-cyclohexyl]-acetic acid;
(viii) {1-[(2,2-dimethyl-propionylamino)-methyl]-cyclohexyl}-acetic acid;
(ix) [1-(phenylacetylamino-methyl)-cyclohexyl]-acetic acid;
(x) [1-(benzoylamino-methyl)-cyclohexyl]-acetic acid benzyl ester;
(xi) [1-(benzoylamino-methyl)-cyclohexyl]-acetic acid phenyl ester;
(xii) [1-(benzoylamino-methyl)-cyclohexyl]-acetic acid ethyl ester;
(xiii) [1-(benzoylamino-methyl)-cyclohexyl]-acetic acid isopropyl ester;
(xiv) [1-(phenylacetylamino-methyl)-cyclohexyl]-acetic acid benzyl ester
(xv) {1-[(2,2-dimethyl-propionylamino)-methyl]-cyclohexyl}-acetic acid benzyl ester;
(xvi) benzoic acid 2-[(1-ethoxycarbonylmethyl-cyclohexylmethyl)-carbamoyl]-benzyl ester.

Various methods may be used to prepare compounds according to the invention. For example, (acyloxy)alkyl carbamate prodrugs of gabapentin may be prepared according to Reaction Scheme I below:

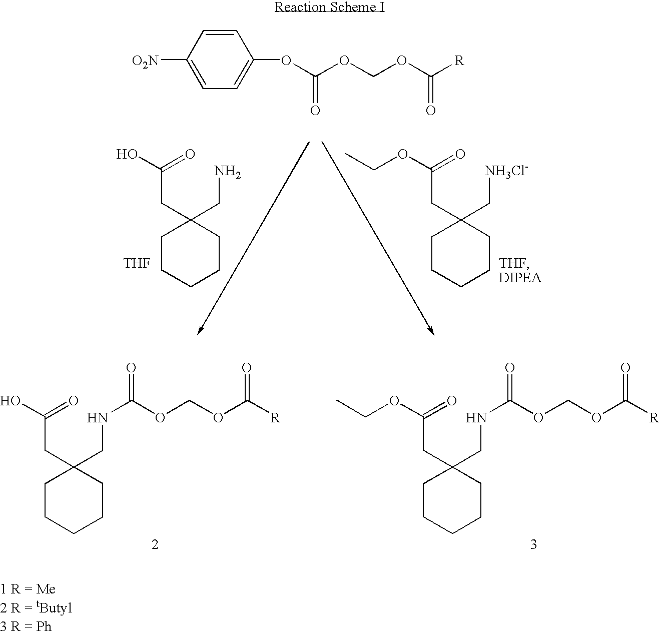

An amino acid to be converted into a pro-drug is reacted with a p-nitrophenyl carbonate ester at ambient temperatures in an inert organic solvent, e.g. an ether solvent such as tetrahydrofuran (THF). In the case of an ester starting material, a salt of said starting material e.g. the chloride and p-nitrophenyl carbonate ester may be reacted in the presence of an organic base e.g. di-isopropylethylamine (DIPEA) in an inert organic solvent at ambient temperatures. An ether (e.g. tetrahydrofuran) may be used as the solvent.

Amide prodrugs of gabapentin may be prepared by reaction scheme II:

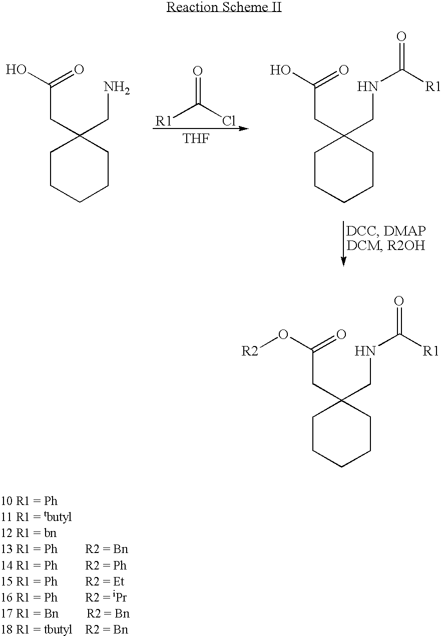

An amino acid starting material is reacted with an acid chloride in an inert organic solvent, e.g. an ether solvent such as tetrahydrofuran at ambient temperatures. If desired the carboxylic acid group may then be esterified by reaction with an alcohol (R2OH) in the presence of dicyclohexylcarbodiimide (DCM) and dimethylaminopyridine (DMAP) at ambient temperatures in an inert solvent such as tetrahydrofuran (THF).

o-(Benzoyloxymethyl)phenyl amide prodrugs of gabapentin may be prepared by reaction scheme III

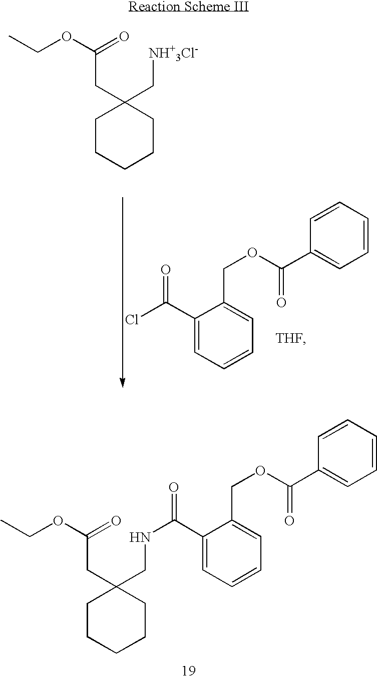

The starting material in the form of a salt e.g. the chloride is reacted with 2-benzoyloxymethyl benzoyl chloride at a temperature below ambient in the presence of a base such as diisopropylethylamine.

The above methods are equally applicable for the preparation of five-membered ring compounds.

The compounds of the invention are expected to be useful in the prevention or treatment of epilepsy and as a mimetic agent for neurodegenerative disorders. Such neurodegenerative disorders are, for example, Alzheimer's disease, Huntington's disease, Parkinson's disease, and Amyotrophic Lateral Sclerosis. The present invention also covers preventing or treating neurodegenerative disorders termed acute brain injury with compounds according to the invention. These include but are not limited to: stroke, head trauma, and asphyxia. Stroke refers to a cerebral vascular disease and may also be referred to as a cerebral vascular incident (CVA) and includes acute thromboembolic stroke. Stroke includes both focal and global ischemia. Also, included are transient cerebral ischemic attacks and other cerebral vascular problems accompanied by cerebral ischemia such as in a patient undergoing carotid endarterectomy specifically or other cerebrovascular or vascular surgical procedures in general, or diagnostic vascular procedures including cerebral angiography and the like. Other incidents are head trauma, spinal cord trauma, or injury from general anoxia, hypoxia, hypoglycemia, hypotension as well as similar injuries seen during procedures from embole, hyperfusion, and hypoxia. The instant invention would be useful in a range of incidents, for example, during cardiac bypass surgery, in incidents of intracranial hemorrhage, in perinatal asphyxia, in cardiac arrest, and status epilepticus. A skilled physician will be able to determine the appropriate situation in which subjects are susceptible to or at risk of, for example, stroke as well as suffering from stroke for administration by methods of the present invention.

The compounds of the invention are also expected to be useful in the prevention or treatment of depression. Depression can be the result of organic disease, secondary to stress associated with personal loss, or idiopathic in origin. There is a strong tendency for familial occurrence of some forms of depression suggesting a mechanistic cause for at least some forms of depression. The diagnosis of depression is made primarily by quantification of alterations in patients' mood. These evaluations of mood are generally performed by a physician or quantified by a neuropsychologist using validated rating scales, such as the Hamilton Depression Rating Scale or the Brief Psychiatric Rating Scale. Numerous other scales have been developed to quantify and measure the degree of mood alterations in patients with depression, such as insomnia, difficulty with concentration, lack of energy, feelings of worthlessness, and guilt. The standards for diagnosis of depression as well as all psychiatric diagnoses are collected in the Diagnostic and Statistical Manual of Mental Disorders (Fourth Edition) referred to as the DSM-IV-R manual published by the American Psychiatric Association, 1994. The compounds of the instant invention are also expected to be useful in the prevention or treatment of anxiety and of panic as demonstrated by means of standard pharmacological procedures.

The compounds of the invention are also expected to be useful in the prevention or treatment of pain. Pain refers to acute as well as chronic pain. Acute pain is usually short-lived and is associated with hyperactivity of the sympathetic nervous system. Examples are postoperative pain and allodynia. Chronic pain is usually defined as pain persisting from 3 to 6 months and includes somatogenic pains and psychogenic pains. Other pain is nociceptive. Still other pain is caused by injury or inflammation of peripheral sensory nerves. It includes, but is not limited to pain from peripheral nerve trauma, herpes virus infection, diabetes mellitus, causalgia, plexus avulsion, neuroma, limb amputation, and vasculitis. Neuropathic pain is also caused by nerve damage from chronic alcoholism, human immunodeficiency virus infection, hypothyroidism, uremia, or vitamin deficiencies. Neuropathic pain includes, but is not limited to pain caused by nerve injury such as, for example, the pain diabetics suffer from. Psychogenic pain is that which occurs without an organic origin such as low back pain, atypical facial pain, and chronic headache. Other types of pain are: inflammatory pain, osteoarthritic pain, trigeminal neuralgia, cancer pain, diabetic neuropathy, restless leg syndrome, acute herpetic and postherpetic neuralgia, causalgia, brachial plexus avulsion, occipital neuralgia, gout, phantom limb, burn, and other forms of neuralgia, neuropathic and idiopathic pain syndrome. The compounds of the invention are also expected to be useful in the prevention or treatment of digestive disorders such as visceral pain, pain associated with cancer, the irritable bowel syndrome, infection and inflammation.

The compounds of the invention can be prepared and administered in a wide variety of oral and parenteral dosage forms. Thus, they can be administered by injection, that is, intravenously, intramuscularly, intracutaneously, subcutaneously, intraduodenally, or intraperitoneally. Also, they can be administered by inhalation, for example, intranasally. Additionally, the compounds of the present invention can be administered transdermally. It will be obvious to those skilled in the art that the following dosage forms may comprise as the active component, either a compound of the invention or a corresponding pharmaceutically acceptable salt.

For preparing pharmaceutical compositions from the present compounds, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted, and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection liquid preparations can be formulated in solution in aqueous polyethylene glycol.

Aqueous solutions suitable for oral use can be prepared by dissolving the active component in water and adding suitable colorants, flavors, stabilizing and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1 g according to the particular application and the potency of the active component. In medical use the drug may be administered three times daily as, for example, capsules of 100 or 300 mg. The composition can, if desired, also contain other compatible therapeutic agents.

In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred. The dosages, however, may be varied depending upon the requirements of the patient, the severity of the condition being treated, and the compound being employed. Determination of the proper dosage for a particular situation is within the skill of the art. Generally, prevention or treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

### Preparation of Reagents

### Acetoxymethyl p-nitrophenyl carbonate (1)

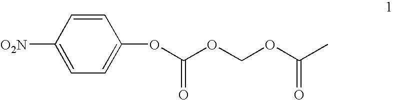

Carbonate 1 was prepared as described in J. Med. Chem, 1988, 31, 318-322 (5.29 g, 98%). Its characteristics were described in J. Org. Chem, 1997, 62, 1356-1362.

νₘₐₓ(film)/cm⁻¹ 1776 (C═O), 1526 (C═C, Ar). δ_{H}(400 MHz; CDCl₃) 2.19 (3H, s, CH₃), 5.88 (2H, s, OCH₂O), 7.42 (2H, d, J 9.6, p-NO₂ArH), 8.30 (2H, d, J 9.2, p-NO₂ArH).

### 2,2-dimethylpropionyloxymethyl p-nitrophenyl carbonate (2)

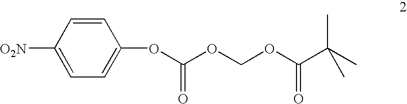

Carbonate 2 was also prepared as described in the above paper (1.16 g, 60%).

νₘₐₓ(film)/cm⁻¹ 1779, 1759 (C═O), 1530 (C═C, Ar). δ_{H}(400 MHz; CDCl₃) 1.26 (9H, s, ^{t}butyl), 5.89 (2H, s, OCH₂O), 7.41 (2H, d, J 9.4, p-NO₂ArH), 8.30 (2H, d, J 9.2, p-NO₂ArH).

### Benzoyloxymethyl p-nitrophenyl carbonate (3)

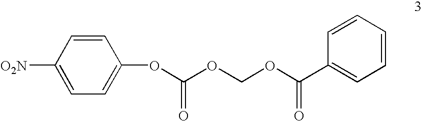

Carbonate 3 was also prepared as described in the above paper (1.76 g, 85%).

νₘₐₓ(film)/cm⁻¹ 1778, 1740 (C═O), 1525 (C═C Ar). δ_{H}(400 MHz; CDCl₃) 6.14 (2H, s, OCH₂O), 7.42 (2H, d, J 9.2,p-NO₂ArH), 7.49 (2H, t, J 8.0, ArH), 7.64 (1H, t, J 7.6, ArH), 8.12 (2H, d, J 7.2, ArH) 8.29 (2H, d, J 9.2, p-NO₂ArH).

The invention will now be further described with reference to the following Examples.

### EXAMPLE 1

### [1-(Acetoxymethoxycarbonylamino-methyl)-cyclohexyl]-acetic acid

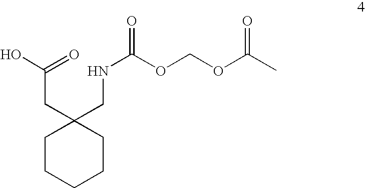

The carbonate 1 (0.4 g, 1.57 mmol) and gabapentin (0.268 g, 1.57 mmol) were stirred in THF (60 ml) at room temperature for 48 hours. The reaction mixture was taken up in ethyl acetate (250 ml) and washed with water (200 ml), 1N HCl (200 ml), dried (MgSO₄) and concentrated in vacuo. The residue was purified by column chromatography (SiO₂, heptane-ethyl acetate, 1:1) to give 4 (0.16 g, 35%).

νₘₐₓ(film)/cm⁻¹ 1725 (C═O). δ_{H}(400 MHz; CDCl₃) 1.34-1.60 (10H, m, cyclohexyl), 2.13 (3H, s, OMe), 2.35 (2H, s, CH₂COOH), 3.27 (2H, d, J 6.8, CH₂NH), 5.37 (1H, bt, NH), 5.74 & 5.78 (2H, 2×s, OCH₂O).

### EXAMPLE 2

### [1-(Acetoxymethoxycarbonylamino-methyl)-cyclohexyl]-acetic acid ethyl ester

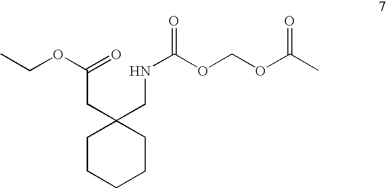

The carbonate 1 (0.4 g, 1.57 mmol), di-isopropylethylamine (0.28 ml, 1.57 mmol) and the hydrogen chloride salt of gabapentin ethyl ester (0.37 g, 1.57 mmol) were stirred in THF (60 ml) at room temperature for 24 hours. The reaction mixture was taken up in ethyl acetate (250 ml) and washed with saturated sodium carbonate (3×500 ml), brine (200 ml), dried (MgSO₄) and concentrated in vacuo. The residue was purified by column chromatography (SiO₂, heptane-ethyl acetate, 1:0 to 8:2) to give 7 (0.29 g, 58%).

νₘₐₓ(film)/cm⁻¹ 1729 (C═O). δ_{H}(400 MHz; CDCl₃) 1.26 (3H, t, J 7.2, COOCH₂CH₃), 1.31-1.60 (10H, m, cyclohexyl), 2.11 (3H, s, COMe), 2.28 (2H, s, CH₂COOEt), 3.23 (2H, d, J 6.8, CH₂NH), 4.14 (2H, q, J 7.2, COOCH₂CH₃), 5.52 (1H, bt, NH), 5.73 (2H, s, OCH₂O).

### EXAMPLE 3

### 2,2-Dimethyl-propionic acid 1-carboxymethylcyclohexylmethylcarbamoyloxymethyl ester

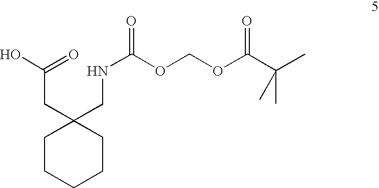

Compound 5 was prepared as was compound 4 from gabapentin and carbonate 2 (0.25 g, 56.4%).

νₘₐₓ(film)/cm⁻¹ 1745, 1715 (C═O). δ_{H}(400 MHz; CDCl₃) 1.22 (9H, s, t-butyl) 1.30-2.00 (10H, m, cyclohexyl), 2.32 (2H, s, CH₂COOH), 3.27 (2H, d, J 6.8, CH₂NH), 5.35 (1H, bt, NH), 5.74 (2H, s, OCH₂O).

### EXAMPLE 4

### 2,2-Dimethyl-propionic acid 1-ethoxycarbonylmethylcyclohexylmethylcarbamoyloxymethyl ester

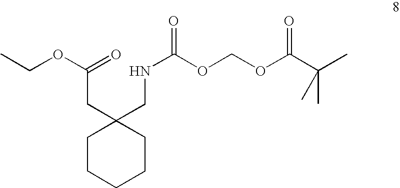

Compound 8 was prepared as described in relation to compound 7 from the hydrogen chloride salt of gabapentin ethyl ester and carbonate 2 (0.29 g, 61%).

νₘₐₓ(film)/cm⁻¹ 1753, 1731 (C═O). δ_{H}(400 MHz; CDCl₃) 1.21 (9H, s, t-butyl), 1.26 (3H, t, J 7.2, COOCH₂CH₃), 1.30-1.60 (10H, m, cyclohexyl), 2.27 (2H, s, CH₂COOEt), 3.23 (2H, d, J 6.8, CH₂NH), 4.13 (2H, q, J 7.2, COOCH₂CH₃), 5.46 (1H, bt, NH), 5.73 (2H, s, OCH₂O).

### EXAMPLE 5

### Benzoic acid 1-carboxymethyl-cyclohexylmethylcarbamoyloxymethyl ester

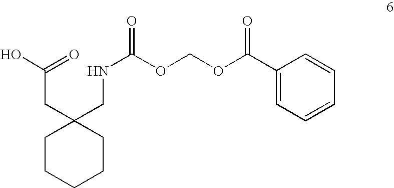

Compound 6 was prepared as was compound 4 from gabapentin and carbonate 3 (0.166 g, 32%).

νₘₐₓ(film)/cm⁻¹ 1737 (C═O). δ_{H}(400 MHz; CDCl₃) 1.31-1.60 (10H, m, cyclohexyl), 2.33 (2H, s, CH₂COOH), 3.27 (2H, d, J 6.8, CH₂NH), 5.39 (1H, bt, NH), 6.00 & 6.04 (2H, s, OCH₂O), 7.46 (2H, t, J 8.0, ArH), 7.60 (1H, t, J 7.6, ArH), 8.08 (2H, d, J 7.6, ArH).

### EXAMPLE 6

### Benzoic acid 1-ethoxycarbonylmethyl-cyclohexylmethylcarbamoyloxymethyl ester

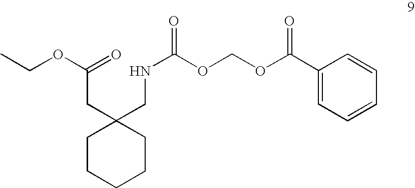

Compound 9 was prepared as was compound 7 from the hydrogen chloride salt of gabapentin ethyl ester and carbonate 3 (0.35 g, 59%).

νₘₐₓ(film)/cm⁻¹ 1736 (C═O). δ_{H}(400 MHz; CDCl₃) 1.24 (3H, t, J 7.2, COOCH₂CH₃), 1.25-1.60 (10H, m, cyclohexyl), 2.27 (2H, s, CH₂COOEt), 3.24 & 3.19 (2H, d, J 6.8, CH₂NH), 4.12 (2H, q, J 6.8, COOCH₂CH₃), 5.53 (1H, t, J 6.4, NH), 6.00 & 6.50 (2H, s, OCH₂O). 7.45 (2H, t, J 8.0, ArH), 7.59 (1H, t, J 7.6, ArH), 8.09 (2H, d, J 7.2, ArH).

### EXAMPLE 7

### [1-(Benzoylamino-methyl)-cyclohexyl]-acetic acid

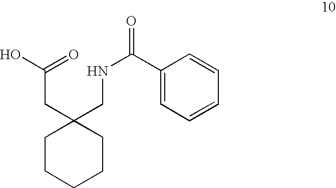

To a stirred suspension of gabapentin (6.0 g, 35 mmol) in THF (80 ml) at room temperature under argon was added benzoyl chloride (4.88 ml, 42 mmol) and the reaction mixture was stirred for 18 hours. The reaction mixture was filtered and concentrated in vacuo. The residue was chromatographed (SiO₂, heptane-ethyl acetate, 1:1 to 3:7) to give 10 (6.03 g, 63%).

νₘₐₓ(film)/cm⁻¹ 1712, 1622 (C═O). δ_{H}(400 MHz; CDCl₃) 1.37-1.60 (10H, m, cyclohexyl), 2.40 (2H, s, CH₂COOH), 3.51 (2H, d, J 6.8, CH₂NH), 6.80 (1H, bt, NH), 7.47 (2H, t, J 8.0, ArH), 7.55 (1H, t, J 7.2, ArH), 7.81 (2H, d, J 7.2, ArH).

### EXAMPLE 8

### {1-[(2,2-Dimethyl-propionylamino)-methyl]-cyclohexyl}-acetic acid

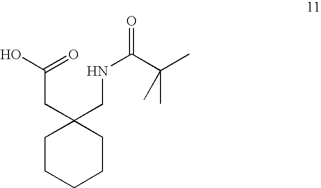

Compound 11 was prepared as described in relation to Compound 10 (0.23 g, 52%).

νₘₐₓ(film)/cm⁻¹ 1715, 1613 (C═O). δ_{H}(400 MHz; CDCl₃) 1.25 (9H, s, ^{t}butyl) 1.25-1.60 (10H, m, cyclohexyl), 2.25 (2H, s, CH₂COOH), 3.27 (2H, d, J 6.8, CH₂N), 6.20 (1H, bt, NH).

### EXAMPLE 9

### [1-(Phenylacetylamino-methyl)-cyclohexyl]-acetic acid

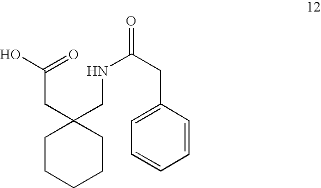

Compound 12 was prepared as described in relation to compound 10 (1.07 g, 21%).

νₘₐₓ(film)/cm⁻¹ 2926 (OH), 1714, 1626 (C═O). δ_{H}(400 MHz; CDCl₃) 1.16-1.56 (10H, m, cyclohexyl), 2.22 (2H, s, CH₂COOH), 3.23 (2H, d, J 7.2, CH₂NH), 3.66 (2H, s, ArCH₂CO), 5.90 (1H, bt, NH), 7.29-7.42 (5H, m, ArH).

### EXAMPLE 10

### [1-(Benzoylamino-methyl)-cyclohexyl]-acetic acid benzyl ester

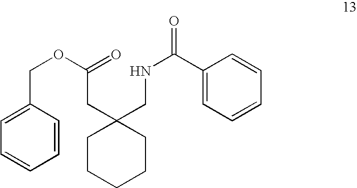

To a stirred mixture of the acid 10 (3.0 g, 11 mmol), 1,3-dicyclohexylcarbodiimide (2.25 g, 11 mmol), and 4-dimethylaminopyridine (1.33 g, 11 mmol) in dichloromethane (80 ml) was added benzyl alcohol (1.19 g, 11 mmol) and the mixture was stirred for 18 hours. The reaction mixture was concentrated in vacuo to 30 ml, filtered and concentrated in vacuo. The residue was chromatographed (SiO₂, heptane-ether, 1:0 to 85:15) to give 13 (2.85 g, 36%).

νₘₐₓ(film)/cm⁻¹ 1731, 1650 (C═O). δ_{H}(400 MHz; CDCl₃) 1.38-1.67 (10H, m, cyclohexyl), 2.43 (2H, s, CH₂COO), 3.46 (2H, d, J 6.8, CH₂NH), 5.15 (2H, s, ArCH₂O), 7.14 (1H, bt, NH), 7.35 (5H, bs, ArH), 7.41 (2H, t, J 7.6, ArH), 7.46-7.52 (1H, m, ArH), 7.74 (2H, d, J 7.2, ArH).

The above compound when administered as a single 5 mg/kg dose PO to rats produced a plasma concentration of gabapentin similar to that shown by gabapentin administered alone, but with a half-life extended from about 1.2 hrs to about 6 hrs.

### EXAMPLE 11

### [1-(Benzoylamino-methyl)-cyclohexyl]-acetic acid phenyl ester

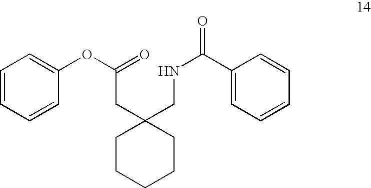

Compound 14 was prepared as described in relation to compound 13 (0.19 g, 38%).

νₘₐₓ(film)/cm⁻¹ 1754, 1650 (C═O). δ_{H}(400 MHz; CDCl₃) 1.43-1.76 (10H, m, cyclohexyl), 2.64 (2H, s, CH₂COO), 3.61 (2H, d, J 6.8, CH₂NHCO),), 7.08 (3H, d, J 8.0, ArH), 7.27 (1H, m, ArH), 7.37-7.43 (4H, m, 3ArH & NH) 7.47 (1H, d, J 7.2, ArH), 7.80 (2H, d, J 7.2, ArH).

### EXAMPLE 12

### [1-(Benzoylamino-methyl)-cyclohexyl]-acetic acid ethyl ester

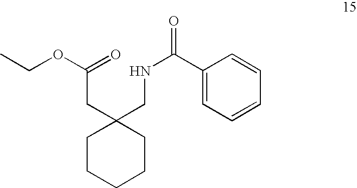

Compound 15 was prepared as described in relation to compound 13 (0.475 g, 62%).

νₘₐₓ(film)/cm⁻¹ 1728, 1644 (C═O). δ_{H}(400 MHz; CDCl₃) 1.28 (3H, t, J 7.2, COOCH₂CH₃), 1.39-1.69 (10H, m, cyclohexyl), 2.39 (2H, s, CH₂COOEt), 3.50 (2H, d, J 6.4, CH₂NH), 4.18 (2H, q, J 7.2, COOCH₂CH₃), 7.29 (1H, bs, NH), 7.40-7.52 (3H, m, ArH), 7.84 (2H, d, J 6.8, ArH).

### EXAMPLE 13

### [1-(Benzoylamino-methyl)-cyclohexyl]-acetic acid isopropyl ester

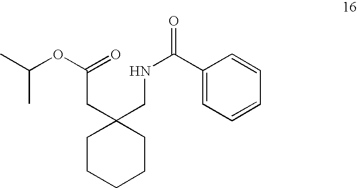

Compound 16 was prepared as described in relation to compound 13 (0.49 g, 60%).

νₘₐₓ(film)/cm⁻¹ 1724, 1645 (C═O). δ_{H}(400 MHz; CDCl₃) 1.26 (6H, d, J 6.2, CH(CH₃)₂), 1.40-1.67 (10H, mm, cyclohexyl), 2.36 (2H, s, CH₂COOⁱPr), 3.49 (2H, d, J 6.8, CH₂NH), 5.06 (1H, septet, J 6.2, CH(CH₃)₂), 7.33 (1H, bt, NH), 7.42-7.52 (3H, m, ArH), 7.84 (2H, d, J 8.0, ArH).

### EXAMPLE 14

### [1-(Phenylacetylamino-methyl)-cyclohexyl]-acetic acid benzyl ester

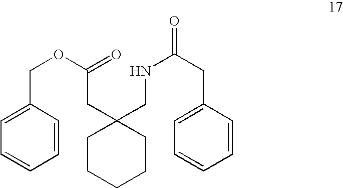

Compound 17 was prepared as described in relation to compound 13 from compound 12 (0.61 g, 58%).

νₘₐₓ(film)/cm⁻¹ 1730, 1645 (C═O) δ_{H}(400 MHz; CDCl₃) 1.20-1.54 (10H, m, cyclohexyl), 2.13 (2H, s, CH₂COO), 3.18 (2H, d, J 6.8, CH₂NH), 3.53 (2H, s, NHCOCH₂), 4.99 (2H, s, ArCH₂O), 6.04 (1H, bt, NH), 7.22-7.39 (10H, m, ArH).

### EXAMPLE 15

### {1-[(2,2-Dimethyl-propionylamino)-methyl]-cyclohexyl}-acetic acid benzyl ester

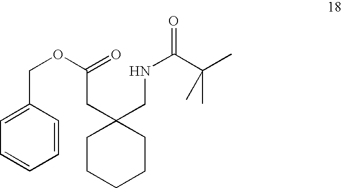

Compound 18 was prepared as described in relation to compound 13 from compound 11 (0.17 g, 53%).

νₘₐₓ(film)/cm⁻¹ 1722, 1661 (C═O); δ_{H}(400 MHz; CDCl₃) 1.15 (9H, s, ^{t}butyl), 1.23-1.60 (10H, m, cyclohexyl), 2.33 (2H, s, CH₂COO), 3.24 (2H, d, J 6.4, CH₂NH), 5.12 (2H, s, ArCH₂O), 6.49 (1H, bt, NH), 7.33-7.38 (5H, m, ArH).

### EXAMPLE 16

### Benzoic acid 2-[(1-ethoxycarbonylmethyl-cyclohexylmethyl)-carbamoyl]-benzyl ester

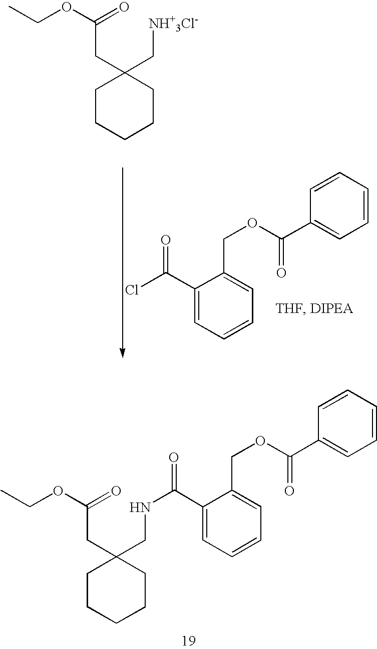

Di-isopropylethyl amine (0.15 ml, 0.85 mmol) was added dropwise to a stirred solution of gabapentin ethyl ester hydrogen chloride salt (0.20 g, 0.85 mmol) in THF (50 ml) under argon at −10° C., followed by a solution of 2-benzoyloxymethyl benzoyl chloride (0.23 g, 0.85 mmol) in THF (20 ml). The reaction mixture was stirred for 5 hours at room temperature and then concentrated in vacuo. The residue was chromatographed (SiO₂, heptane-ethyl acetate, 1:1 to 8:2) to give 19 (0.23 g, 61%).

νₘₐₓ(film)/cm⁻¹ 1720, 1655 (C═O). δ_{H}(400 MHz, CDCl₃) 1.23 (3H, t, J 7.2, COOCH₂CH₃), 1.38-1.82 (10H, m, cyclohexyl), 2.34 (2H, s, CH₂COOEt), 3.47 (2H, d, J 6.4, CH₂NH), 4.10 (2H, q, J 7.2, COOCH₂CH₃), 5.62 (2H, s, CH₂OCOAr), 6.89 (1H, bt, NH), 7.36-7.46 (4H, m, ArH), 7.51-7.57 (3H, m, ArH), 8.07 (2H, d, J 7.2, ArH).

1. A pharmaceutical composition comprising a pharmaceutically acceptable carrier and a therapeutically effective amount of a compound of the formula (I) or (II) 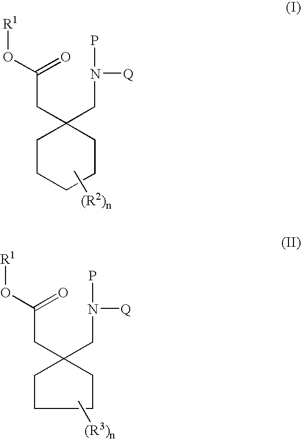
wherein:
n is 0, 1 or 2;
P represents hydrogen or methyl;
Q represents a labile amine- or amide-forming organic group that becomes removed in the human or animal body, and in a compound of formula (I) is other than acyl;
R¹ represents hydrogen or a labile ester-forming group selected from substituted and unsubstituted C₁-C₆ alkyl, benzyl and phenyl groups that become removed in the human or animal body;
R² represents methyl; and
the groups R³ (which when n is 2 may be the same or different) represent C₁-C₆ alkyl, or a pharmaceutically acceptable salt thereof.

2. The composition of claim 1, wherein the compound is of the formula (IIIa)-(IIIc) 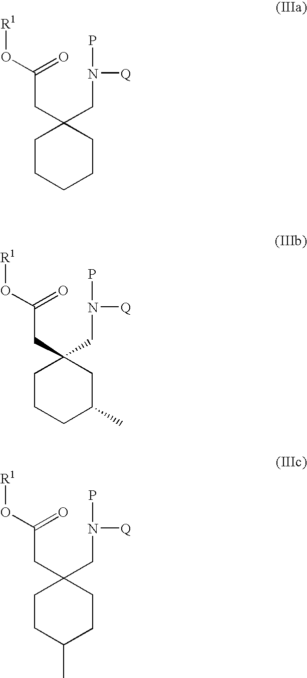
in which R¹, P and Q are as defined in claim 1.

3. The composition of claim 1, wherein the compound is of the formula (IV), (V), (VI) or (VII) 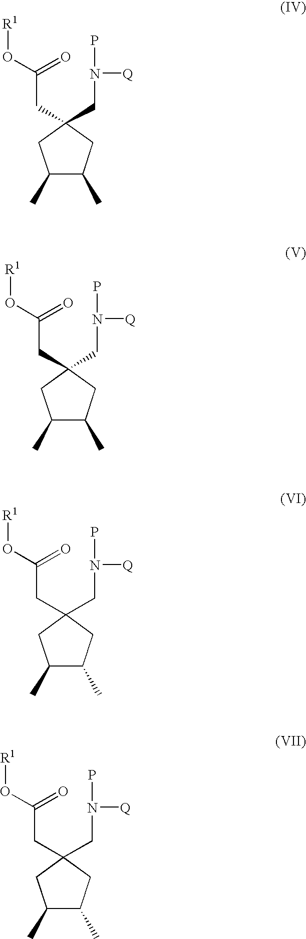
in which R¹, P and Q are as defined in claim 1.

4. The composition of claim 1, wherein the compound is of the formula (VIII) or (IX) 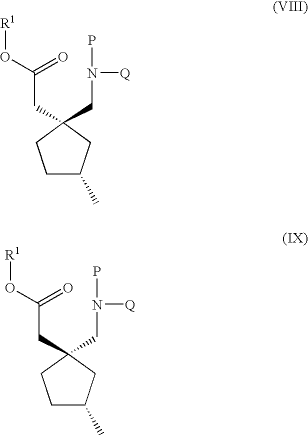
in which R¹, P and Q are as defined in claim 1.

5. The composition of claim 1, wherein:
Q represents a labile amine- or amide-forming organic group that is selected from 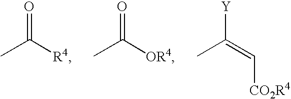 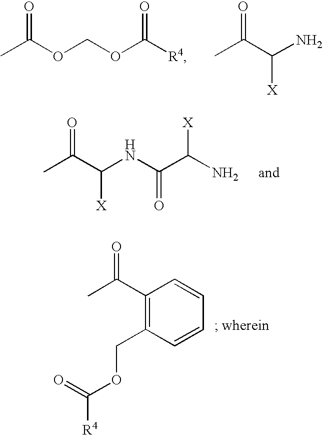
R⁴ represents hydrogen, straight or branched chain C₁-C₆ alkyl, phenyl or benzyl in which the benzene ring may be substituted or unsubstituted;
X represents a phenyl group or any of the side chains of the 20 naturally encoded α-amino acids; and
Y represents hydrogen, straight or branched chain C₁-C₆ alkyl, or —CH₂CO₂R⁵ in which R⁵ represents straight or branched chain C₁-C₆ alkyl.

6. The composition of claim 1, in which R¹ is hydrogen.

7. The composition of claim 1, in which R¹ is other than hydrogen and is more labile than Q.

8. The composition of claim 7, in which R¹ is methyl or t-butyl.

9. The composition of claim 7, wherein R¹ represents ethyl, iso-propyl, phenyl or benzyl.

10. The composition of claim 1, wherein Q is removed hydrolytically.

11. The composition of claim 1, wherein Q is removed enzymatically.

12. The composition of claim 1, wherein R⁴ represents t-butyl, benzyl or phenyl.

13. A pharmaceutical composition comprising a pharmaceutically acceptable carrier and a therapeutically effective amount of any of the compounds below:
[1-(acetoxymethoxycarbonylamino-methyl)-cyclohexyl]-acetic acid;
[1-(acetoxymethoxycarbonylamino-methyl)-cyclohexyl]-acetic acid ethyl ester;
2,2-dimethyl-propionic acid 1-carboxymethylcyclohexylmethyl-carbamoyloxymethyl ester;
2,2-dimethyl-propionic acid 1-ethoxycarbonylmethyl-cyclohexylmethylcarbamoyloxymethyl ester;
benzoic acid 1-carboxymethyl-cyclohexylmethylcarbamoyloxymethyl ester;
benzoic acid 1-ethoxycarbonylmethyl-cyclohexylmethylcarbamoyloxymethyl ester;
[1-(benzoylamino-methyl)-cyclohexyl]-acetic acid;
{1-[(2,2-dimethyl-propionylamino)-methyl]-cyclohexyl}-acetic acid;
[1-(phenylacetylamino-methyl)-cyclohexyl]-acetic acid;
[1-(benzoylamino-methyl)-cyclohexyl]-acetic acid benzyl ester;
[1-(benzoylamino-methyl)-cyclohexyl]-acetic acid phenyl ester;
[1-(benzoylamino-methyl)-cyclohexyl]-acetic acid ethyl ester;
[1-(benzoylamino-methyl)-cyclohexyl]-acetic acid isopropyl ester;
[1-(phenylacetylamino-methyl)-cyclohexyl]-acetic acid benzyl ester;
{1-[(2,2-dimethyl-propionylamino)-methyl]-cyclohexyl}-acetic acid benzyl ester;
benzoic acid 2-[(1-ethoxycarbonylmethyl-cyclohexylmethyl)-carbamoyl]-benzyl ester.

14. A method for preventing or treating epilepsy comprising administering a therapeutically effective amount of a composition according to claim 1 to a mammal in need of said prevention or treatment.

15. A method for preventing or treating faintness attacks, hypokinesia and cranial disorders comprising administering a therapeutically effective amount of a composition according to claim 1 to a mammal in need of said prevention or treatment.

16. A method for preventing or treating neurodegenerative disorders comprising administering a therapeutically effective amount of a composition according to claim 1 to a mammal in need of said prevention or treatment.

17. A method for preventing or treating depression comprising administering a therapeutically effective amount of a composition according to any claim 1 to a mammal in need of said prevention or treatment.

18. A method for preventing or treating anxiety comprising administering a therapeutically effective amount of a composition according to claim 1 to a mammal in need of said prevention or treatment.

19. A method for preventing or treating panic comprising administering a therapeutically effective amount of a composition according to claim 1 to a mammal in need of said prevention or treatment.

20. A method for preventing or treating pain comprising administering a therapeutically effective amount of a composition according to claim 1 to a mammal in need of said prevention or treatment.

21. A method for preventing or treating neuropathological disorders comprising administering a therapeutically effective amount of a composition according to claim 1 to a mammal in need of said prevention or treatment.

22. A method for preventing or treating digestive disorders comprising administering a therapeutically effective amount of a composition according to claim 1 to a mammal in need of said prevention or treatment.

23. A compound of the formula (I) or (II) 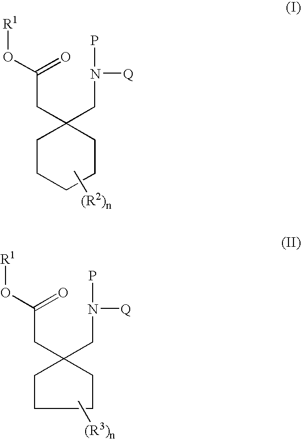
wherein:
n is 0, 1 or 2;
P represents hydrogen or methyl;
Q represents a labile amine- or amide-forming organic group that is selected from 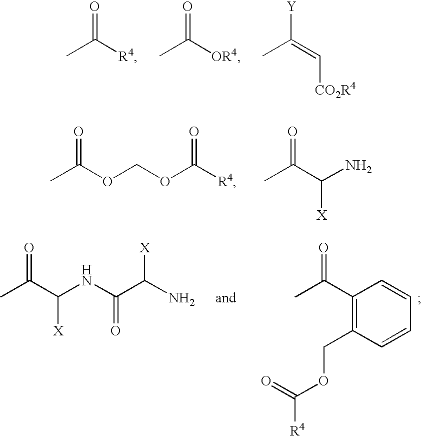
R¹ represents hydrogen or a labile ester-forming group selected from substituted and unsubstituted C₁-C₆ alkyl, benzyl and phenyl groups that become removed in the human or animal body;
R² represents methyl;
the group or groups R³ (which when n is 2 may be the same or different) represent C₁-C₆ alkyl;
R⁴ represents hydrogen, straight or branched chain C₁-C₆ alkyl, phenyl or benzyl in which the benzene ring may be substituted or unsubstituted;
Y represents hydrogen, straight or branched chain C₁-C₆ alkyl, or —CH₂CO₂R⁵ in which R⁵ represents straight or branched chain C₁-C₆ alkyl; and
X represents a phenyl group or any of the side chains of the 20 naturally encoded α-amino acids;
provided that:
(a) in a compound of formula (I) where Q is —COR⁴, R⁴ is not alkyl;
(b) in a compound of formula (I) where Q is —COOR⁴, R⁴ is not alkyl or benzyl; and
(c) in a compound of formula (II) Q is not —COOMe.

24. A compound of the formula (IIIa)-(IIIc) 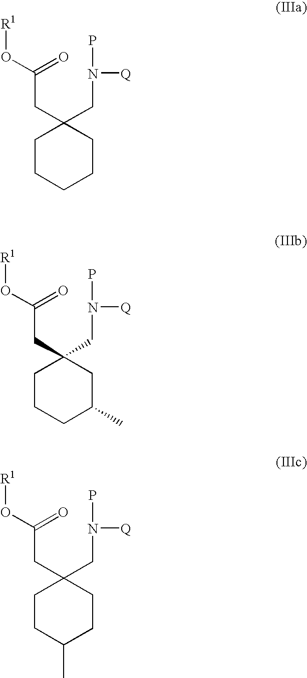
in which R¹, P and Q are as defined in claim 23.

25. A compound of the formula (IV), (V), (VI) or (VII) 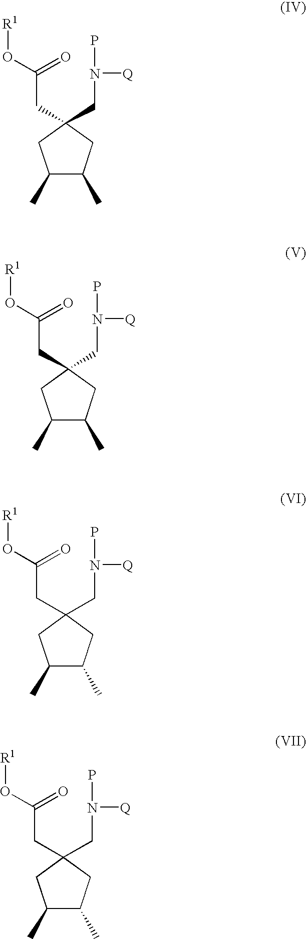
in which R¹, P and Q are as defined in claim 23.

26. A compound of the formula (VIII) or (IX) 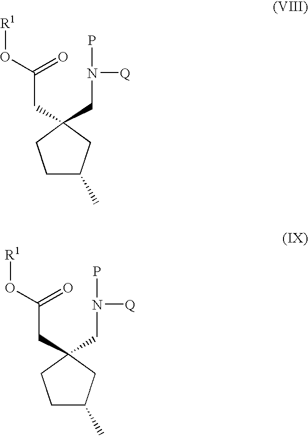
in which R¹, P and Q are as defined in claim 23.

27. The compound of claim 23, in which R¹ is hydrogen.

28. The compound of claim 23, in which R¹ is other than hydrogen and is more labile than Q.

29. The compound of claim 23, in which R¹ is methyl or t-butyl.

30. The compound of claim 23, wherein R¹ represents ethyl, iso-propyl, phenyl or benzyl.

31. The compound of claim 23, wherein Q is removed hydrolytically.

32. The compound of claim 23, wherein Q is removed enzymatically.

33. The compound of claim 23, wherein R⁴ represents t-butyl, benzyl or phenyl.

34. The compound of claim 23, wherein Q is 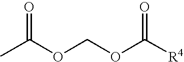
wherein R⁴ represents methyl, t-butyl or phenyl.

35. Any of the compounds below:
[1-(acetoxymethoxycarbonylamino-methyl)-cyclohexyl]-acetic acid;
[1-(acetoxymethoxycarbonylamino-methyl)-cyclohexyl]-acetic acid ethyl ester;
2,2-dimethyl-propionic acid 1-carboxymethylcyclohexylmethyl-carbamoyloxymethyl ester;
2,2-dimethyl-propionic acid 1-ethoxycarbonylmethyl-cyclohexylmethylcarbamoyloxymethyl ester;
benzoic acid 1-carboxymethyl-cyclohexylmethylcarbamoyloxymethyl ester;
benzoic acid 1-ethoxycarbonylmethyl-cyclohexylmethylcarbamoyloxymethyl ester;
[1-(benzoylamino-methyl)-cyclohexyl]-acetic acid;
{1-[(2,2-dimethyl-propionylamino)-methyl]-cyclohexyl}-acetic acid;
[1-(phenylacetylamino-methyl)-cyclohexyl]-acetic acid;
[1-(benzoylamino-methyl)-cyclohexyl]-acetic acid benzyl ester;
[1-(benzoylamino-methyl)-cyclohexyl]-acetic acid phenyl ester;
[1-(benzoylamino-methyl)-cyclohexyl]-acetic acid ethyl ester;
[1-(benzoylamino-methyl)-cyclohexyl]-acetic acid isopropyl ester;
[1-(phenylacetylamino-methyl)-cyclohexyl]-acetic acid benzyl ester;
{1-[(2,2-dimethyl-propionylamino)-methyl]-cyclohexyl}-acetic acid benzyl ester;
benzoic acid 2-[(1-ethoxycarbonylmethyl-cyclohexylmethyl)-carbamoyl]-benzyl ester.

36. A method for making a compound of the formula (I) or (II) above, which comprises:
coupling a compound of the formula: 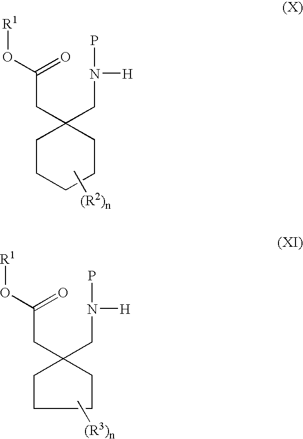
in which P and R¹-R³ have the meanings given in claim 23 and in which said compound is in the form of a free base or an ammonium salt with a compound of the formula 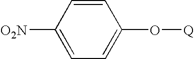
or QCl where Q has the meaning defined above.

37. The method of claim 36, in which the compound (X) or (XI) is a carboxylic acid and comprising the further step of esterifying the carboxyl group with a substituted or unsubstituted C₁-C₆ alkanol, benzyl alcohol or phenol.
